(19) United States
(12) Patent Application Publication
(10) Pub. No.: US 2026/0275425 A1
(43) Pub. Date: Sep. 17, 2026

(54) System and Assay for On-Site Detection and Quantification of Corrosion Causing Microbes

(71) Applicant: Indian Oil Corporation, Mumbai (IN)

(72) Inventors: Prakash Chandra Sahoo, Faridabad (IN); Manoj Kumar, Faridabad (IN); Pragya, Faridabad (IN); Ravi Prakash Gupta, Faridabad (IN); Umish Srivastav, Faridabad (IN)

(73) Assignee: Indian Oil Corporation Limited, Mumbai, MH (IN)

(21) Appl. No.: 19/353,565

(22) Filed: Oct. 8, 2025

(30) Foreign Application Priority Data
Oct. 8, 2024 (IN) 202421076264

(51) Int. Cl. C12Q 1/689

(52) U.S. Cl. CPC C12Q 1/689

(57) ABSTRACT

The present disclosure relates to a real-time polymerase chain reaction based system and assay for rapid and accurate on-site detection of corrosion-causing microbes in diverse industrial settings. The present disclosure provides RT-PCR-reagents comprising agnostic DNA isolation bio-buffer PCR augmenter and PCR reaction medium that enable efficient DNA extraction and amplification with real-time monitoring capabilities, facilitating the efficient identification of corrosion-causing microbes. The present disclosure also relates to an RT-PCR based assay for the on-site detection and quantification of corrosion-causing microbes. The assay entails collecting samples from corroded surfaces or surrounding environments, followed by microbial DNA extraction utilizing a specific DNA isolation bio-buffer, PCR augmenter and PCR buffer composition. During the RT-PCR assay, specific primers targeting conserved genetic regions associated with microbial corrosion are employed for amplification. The assay of the present disclosure is simple making it well-suited for rapid and accurate detection of corrosion causing microbes in real field applications.

This application claims priority to Indian Application No. 202421076264 dated Oct. 8, 2024. The contents of this application are hereby incorporated by reference in its entirety.

The Sequence Listing submitted herewith in XML format via Patent Center, entitled “1348IN2194_seqlisting.xml,” size 14,337 bytes, created on Apr. 14, 2026, is incorporated herein by reference in its entirety.

### FIELD OF THE INVENTION

The present disclosure relates to systems and assays for the detection and quantification of corrosion causing microbes. More particularly, the present disclosure relates to a real-time polymerase chain reaction (RT-PCR) system and assay for on-site detection and quantification of corrosion causing microbes in real field applications.

### BACKGROUND OF THE INVENTION

Corrosion of metallic structures due to microbial activity poses a significant challenge across various industrial sectors, including oil and gas, marine, infrastructure, and water treatment. Microbial-induced corrosion (MIC) is a complex process driven by the metabolic activities of specific microorganisms, leading to accelerated deterioration, safety risks, and substantial economic losses. Early detection and accurate identification of the microbes responsible for corrosion are crucial for implementing timely mitigation strategies and preserving the integrity of metal infrastructure.

Microbiologically-Influenced Corrosion (MIC) accounts for 20% of total corrosion costs. With its ability to degrade the integrity, safety, and reliability of pipeline operations, MIC is a major contributor to the corrosion problem and a leading cause of pipe failure. MIC is becoming an even higher-priority issue for the oil and gas industry with the aging of pipelines and other infrastructure. Oil and gas facilities are particularly susceptible to MIC related problems where there is exposure of metals to bacteria found in water. The problem has intensified in recent years with the growing incidence of reservoir souring caused by the use of water injection treatments for increasing production and transportation of low-quality crude through pipelines.

Fast and accurate monitoring of MIC can help in proper management of pipelines and controlling effective dosing of biocide. In current practice, samples are sent to laboratories and during transportation there are unwanted changes in the microbial abundances of various MIC causing groups. This affects the treatment efficiency and also delays any immediate action, if required to prevent the on-going corrosion due to microbial growth.

Culture dependent methods for detecting and quantifying MIC microbes are less sensitive and time consuming. These shortcomings of culture dependent methods can be overcome by molecular based methodology. One such method is quantitative real time PCR assay (qPCR). qPCR has been widely used to determine the presence of microorganisms in many different types of complex environmental samples such as sediments, water, wastewater, and marine samples.

There are a plethora of systems and methods known in the art for RT-PCR based detection of MIC causing microbes. U.S. Pat. No. 7,384,770 B1 details a method for quickly measuring the levels of acetic acid-producing bacteria using real-time PCR. The said method involves a specific set of primer pairs designed to amplify the nucleic acid of these bacteria. Each primer within the set contains an oligonucleotide that targets the ackA gene region of acetic acid-producing bacteria. These primers are specifically designed to effectively amplify the nucleic acid of acetic acid-producing bacteria found in gas and oil production operations.

U.S. Pat. No. 7,790,865 B1 discusses the development of eluting reagents, methods, and kits for isolating DNA. It describes a solid support system that enables the purification, amplification, and characterization of DNA from both liquid and dried biological samples. The purified DNA obtained through this process is highly suitable for various downstream applications, including widely utilized techniques like amplification and restriction enzyme digestion.

U.S. Pat. No. 5,234,809B2 describes a method for purifying DNA from samples other than complex mixtures like blood. By omitting the elution step, the number of reagents and steps involved in the process can be reduced by one. U.S. Pat. No. 5,496,562 presents a method for purifying cellulose filter paper containing dried blood. This method involves the use of four reagents during four phenol washes and five isopropanol washes. Once dried, a small piece of the filter paper is cut and directly used as a substrate for PCR amplification. Although these methods utilize bound DNA for analysis, they still involve multiple steps and the use of hazardous reagents.

WO1991012342 describes modifications in the mixing of PCR reagents and the initiation of the enzymatic reaction, as well as the replacement of mineral oil, typically used as a vapor barrier to prevent solvent evaporation, with a grease or wax substitute. By utilizing these new mixtures, reagent mixing can be delayed until the initial heating step of PCR amplification. This delay reduces the generation of nonspecific products by the enzymes, which typically occurs when a complete mixture of PCR reagents, with or without the test sample, remains at room temperature or below. Additionally, these mixtures extend the shelf-life of PCR reagents and offer increased protection against contamination by PCR products in the laboratory environment.

CN105925692B presents a PCR premixed liquid that remains stable at room temperature. This invention is specifically related to PCR experiments. The PCR premixed liquid comprises Taq enzyme, buffer, dNTP, as well as stabilizing agents such as glycerol, sodium azide, and 1,2,4-triazoles. By using this PCR premixed liquid, the stability of the mixture is maintained for 144 hours or more at normal room temperature. This significantly improves working efficiency and reduces costs, as there is no need for refrigeration or special storage conditions.

However, the aforesaid prior arts have several limitations like the existing methods commonly involve numerous steps and involve use of potentially hazardous reagents. Further, the uneven distribution of samples, such as muck samples, within the solution makes the process of total DNA extraction for RTPCR laborious. The reagents used are not designed to specifically target the nucleic acid sequences of corrosion-causing microbes. Lack of specificity may result in false-positive or false-negative results. It is crucial to ensure that the reagents and the laboratory environment are free from any contamination, including cross-contamination between samples. Therefore, cannot be used in real field conditions. The stability of the reagents over time is essential for reliable and consistent results. Reagents used in the prior art has limited shelf-life may require frequent replacements, increasing the cost and logistical challenges. Some environmental factors or components present in the sample matrix may also interfere with the PCR reaction, leading to inaccurate results. Compatibility issues may also affect the amplification efficiency and overall performance of the assay. Sometimes, the results obtained from RT-PCR do not correlate with the microbial concentration, which is crucial for analysing the extent of corrosion.

In order to overcome the aforesaid drawbacks of the conventional systems and processes, the present disclosure provides an RT-PCR system and assay for rapid and accurate detection of corrosion-causing microbes in diverse industrial settings. The RT-PCR system of the present disclosure comprises (a) DNA isolation bio-buffer (b) PCR augmenter and (c) PCR reaction medium, which enable efficient DNA extraction and amplification with real-time monitoring capabilities thus facilitating the efficient identification of corrosion-causing microbes. Also, the present disclosure provides a simple and user-friendly assay for rapid and accurate detection of corrosion causing microbe in real field applications, the assay comprises: collecting samples from corroded surfaces or surrounding environments, followed by microbial DNA extraction utilizing a specific DNA isolation buffer and PCR buffer composition. During the RT-PCR assay, specific primers targeting conserved genetic regions associated with microbial corrosion are employed for amplification. The assay can directly count the microbial quantity form RT-PCR results.

The primary object of the present disclosure is to develop a rapid and sensitive assay for on-site detection and quantification of corrosion-causing microbes in diverse industrial settings.

Another object of the present disclosure is to provide an efficient assay for extracting DNA from samples collected from corroded surfaces or surrounding environments.

Yet another object of the present disclosure is to provide a real-time polymerase chain reaction (RT-PCR) assay for the timely identification of corrosion-causing microbes.

Still another object of the present disclosure is to provide a set of primers to effectively target and amplify conserved genetic regions associated with microbial corrosion.

Another object of the present disclosure is to provide an assay for direct counting of microbial quantity from RT-PCR results, eliminating the need for additional steps or calculations for quantification.

The process of the present disclosure is advantageous as it provides an RT-PCR-reagent and assay for real-time polymerase chain reaction (RT-PCR) system. The RT-PCR system of the present disclosure incorporates a DNA isolation bio-buffer and a PCR reaction medium for efficient DNA extraction and amplification. The system of the present disclosure allows for real-time monitoring capabilities, enabling rapid and accurate detection of corrosion-causing microbes. The assay of the present disclosure involves collecting samples from corroded surfaces or surrounding environments. A specific DNA isolation buffer and PCR buffer composition are utilized for microbial DNA extraction. During the RT-PCR assay, specific primers targeting conserved genetic regions associated with microbial corrosion are used for amplification. The assay of the present disclosure allows for direct quantification of microbial quantity from RT-PCR results. Therefore, the system and assay of the present disclosure facilitates rapid and accurate detection of corrosion-causing microbes in diverse industrial settings.

### SUMMARY OF INVENTION

In an aspect of the present disclosure, there is provided a set of primers for on-site detection and quantification of corrosion-causing microorganisms.

In an embodiment of the present disclosure, there is provided a set of PCR primers of SEQ ID NO: 1-15 for on-site detection and quantification of corrosion-causing microorganisms.

In an embodiment of the present disclosure, there is provided a set of PCR primers as described herein, wherein the primers of SEQ ID NO: 1-4 are used to detect and quantify sulfate reducing bacteria (SRB), and wherein a primer of SEQ ID NO: 1 and 3 is a forward primer and a primer of SEQ ID NO: 2 and 4 is a reverse primer.

In an embodiment of the present disclosure, there is provided a set of PCR primers as described herein, wherein the primers of SEQ ID NO: 5-10 are used to detect and quantify AAPB, and wherein a primer of SEQ ID NO: 5, 7 and 9 is a forward primer and a primer of SEQ ID NO: 6, 8 and 10 is a reverse primer.

In an embodiment of the present disclosure, there is provided a set of PCR primers as described herein, wherein the primers of SEQ ID NO: 11-15 are used to detect and quantify BAPB, and wherein a primer of SEQ ID NO: 11 and 12 is a forward primer and a primer of SEQ ID NO: 13, 14 and 15 is a reverse primer.

In an embodiment of the present disclosure, there is provided a set of PCR primers as described herein, wherein the primers are efficient in detecting and quantifying of corrosion-causing microorganisms selected from sulfate reducing bacteria (SRB), AAPB (Acetic Acid producing bacteria) and BAPB (Butyric Acid Producing Bacteria).

In another aspect of the present disclosure, there is provided a real-time polymerase chain reaction (RT-PCR) based assay for on-site detection and quantification of corrosion-causing microbes, said assay comprising:
obtaining a sample from the site affected by corrosion;
isolating DNA from the corrosion sample using a DNA isolation bio-buffer;
stabilizing the isolated DNA using a PCR augmenter;
introducing the stabilized DNA into a PCR reaction medium;
amplifying the stabilized DNA, wherein the amplification step comprises hybridizing an aliquot of DNA using the primers of SEQ ID NO: 1-15; and
detecting and quantifying the amplified product.

In an embodiment of the present disclosure, there is provided a real-time polymerase chain reaction (RT-PCR) based assay as described herein, wherein the sample is collected from sources selected from muck samples taken during pigging; corrosion coupons; samples from cooling towers; or any other metallic surfaces, and wherein the microorganism is selected from sulfate reducing bacteria (SRB), AAPB (Acetic Acid producing bacteria) and BAPB (Butyric Acid Producing Bacteria).

In an embodiment of the present disclosure, there is provided a real-time polymerase chain reaction (RT-PCR) based assay as described herein, wherein the DNA isolation bio-buffer comprises 1-2% TritonX100, 5 mM EDTA, 10 mM Tris HCl, 20 μg/ml Proteinase K, 0.25 mg/ml lysozyme, 2 mg/ml CTAB, 6 mg/ml β-Mercaptoethanol, 3 mg/ml sodium metabisulfite and nuclease free water.

In an embodiment of the present disclosure, there is provided a real-time polymerase chain reaction (RT-PCR) based assay as described herein, wherein the PCR augmenter comprises 1-2% TritonX100, 10 mM 2-pyrrolidone, 2 mM Isobutyramide, 5 mM N-methylacetamide, 2 mg/ml Quantum dots and nuclease free water.

In an embodiment of the present disclosure, there is provided a kit for on-site, rapid testing of corrosion-inducing microorganisms comprising a set of PCR primers of SEQ ID NO: 1-15; a DNA isolation bio-buffer comprising 1-2% TritonX100, 5 mM EDTA, 10 mM Tris HCl, 20 μg/ml Proteinase K, 0.25 mg/ml lysozyme, 2 mg/ml CTAB, 6 mg/ml β-Mercaptoethanol, 3 mg/ml sodium metabisulfite and nuclease free water; a PCR augmenter comprising 1-2% TritonX100, 10 mM 2-pyrrolidone, 2 mM Isobutyramide, 5 mM N-methylacetamide, 2 mg/ml Quantum dots and nuclease free water; a PCR reaction medium; a portable Real-Time Polymerase Chain Reaction (RT-PCR) apparatus; a pipetting device; a nano-scale sample analysis system; a mobile centrifuge unit; a temperature regulation module; and single-use consumable items.

These and other features, aspects, and advantages of the present subject matter will be better understood with reference to the following description. This summary is provided to introduce a selection of concepts in a simplified form.

### BRIEF DESCRIPTION OF THE ACCOMPANYING DRAWINGS

The following FIGURES form part of the present specification and are included to further illustrate aspects of the present disclosure. The disclosure may be better understood by reference to the FIGURES in combination with the detailed description of the specific embodiments presented herein.

FIG. 1 is a graphical representation of standard curve for DNA copies corresponding to the Ct value.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps of the process, features of the product, referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described. All publications mentioned herein are incorporated herein by reference.

The present disclosure is not to be limited in scope by the specific embodiments described herein, which are intended for the purposes of exemplification only. Functionally equivalent products and methods are clearly within the scope of the disclosure, as described herein.

The present disclosure relates to a system and a rapid and sensitive assay for detection and quantification of corrosion-causing microbes in diverse industrial settings.

In an aspect, the present disclosure relates to a set of primers to effectively target and amplify conserved genetic regions associated with microbial corrosion.

In an embodiment of the present disclosure, there is provided a set of primers of SEQ ID NO: 1-15 for the amplification of conserved genetic regions of microbes associated with corrosion.

In a preferred embodiment, the set of primers of SEQ ID NO:1-15 amplify the conserved genetic regions of sulphur reducing bacteria (SRB), and acid producing microbes namely AABP (Acetic Acid producing bacteria) and BAPB (Butyric Acid Producing Bacteria)

In an embodiment of the present disclosure, there is provided a set of primers of SEQ ID NO: 1-15 as described herein, wherein primer of SEQ ID NO: 1-4 are used to detect and quantify sulfate reducing bacteria (SRB).

In an embodiment of the present disclosure, there is provided a set of primers of SEQ ID NO: 1-4 for detecting and quantifying sulfate reducing bacteria (SRB), wherein primer of SEQ ID NO: 1 and 3 is a forward primer and primer of SEQ ID NO: 2 and 4 is a reverse primer.

In an embodiment of the present disclosure, there is provided a set of primers of SEQ ID NO:1-15 as described herein, wherein primer of SEQ ID NO: 5-10 are used to detect and quantify AAPB and wherein a primer selected from SEQ ID NO: 5, 7 and 9 is a forward primer and a primer selected from SEQ ID NO: 6, 8 and 10 is a reverse primer.

In an embodiment of the present disclosure, there is provided a set of primers of SEQ ID NO:1-15 as described herein, wherein the primers of SEQ ID NO: 11-15 are used to detect and quantify BAPB, and wherein a primer selected from SEQ ID NO: 11 and 12 is a forward primer and a primer selected from SEQ ID NO: 13, 14 and 15 is a reverse primer.

In another aspect, the present disclosure relates to a real-time polymerase chain reaction (RT-PCR) assay for identifying corrosion-causing microbes, the process comprises:
obtaining a sample from the site affected by corrosion;
isolating DNA from the corrosion sample utilizing a DNA isolation bio-buffer;
stabilizing the isolated DNA using a PCR augmenter;
introducing the stabilized DNA into the PCR reaction medium along with primers of SEQ ID NO: 1-15;
performing PCR analysis to amplify and detect specific microbial DNA sequences;
correlating the PCR analysis results with the bacterial count and providing recommendations for biocide treatment.

In an embodiment, the present disclosure provides a real-time polymerase chain reaction (RT-PCR) based assay for the on-site detection and quantification of corrosion-causing microbes, the method comprises:
obtaining a sample from the site affected by corrosion;
isolating DNA from the corrosion sample using a DNA isolation bio-buffer;
stabilizing the isolated DNA using a PCR augmenter;
introducing the stabilized DNA into a PCR reaction medium;
amplifying the stabilised DNA, wherein the amplification step comprises hybridizing an aliquot of DNA using the primers of SEQ ID NO: 1-15;
detecting and quantifying the amplified product.

In an embodiment, the present disclosure provides a RT-PCR assay for identifying corrosion-causing microbes as described herein, wherein the sample is collected from sources selected from muck samples taken during pigging, corrosion coupons, samples from cooling towers, or any other metallic surfaces. The samples, once collected, are placed within a sterile container.

In an embodiment, the present disclosure provides an RT-PCR assay for identifying corrosion-causing microbes as described herein, wherein the sample is selected from muck sample, water sample and coupons. The sample was collected using a spatula (~0.1 g) to 0.5 ml clear PCR tube, followed by brief centrifugation (30 s) at 10000-12000 rpm such that it settles at the bottom.

In an embodiment, the present disclosure provides an RT-PCR assay for identifying corrosion-causing microbes as described herein, wherein the microorganism is selected from sulfate reducing bacteria (SRB), Acetic Acid producing bacteria (AAPB) and Butyric Acid Producing Bacteria (BAPB)

In an embodiment, the isolation of DNA from the corrosion sample is carried out utilizing a DNA isolation bio-buffer, as mentioned in table 1 below:

TABLE 1
Composition of DNA isolation bio-buffer (5 ml)

| Chemical | components |
|---|---|
| Triton x100 | 1-2% |
| EDTA | 5-mM |
| TRIS-HCl (pH-8.0) | 10 mM |
| Proteinase K | 20 μg/ml |
| Lysozyme | 0.25 mg/ml |
| CTAB | 2 mg/ml |
| β-Mercaptoethanol | 6 mg/ml |
| Sodium metabisulfite | 3 mg/ml |
| Nuclease-free Water | To make the volume to 5 ml |

In an embodiment, 100-120 μL of DNA isolation bio-buffer was added to 0.5 ml PCR tube containing muck sample.

The sample after addition of DNA isolation bio-buffer was kept on dry bath for 7-10 minutes at 90° C. for sample lysis. The lysed sample tube was taken out of dry bath and centrifuged for 30 seconds at 10,000-12,000 rpm to settle down the lysed sample particles to the bottom of the tube. The clear sample was transferred to a 0.5 ml clear PCR tube.

In an embodiment, after isolation of DNA, its stabilization is achieved by using a PCR augmenter composition, as shown in below table 2. The sample was taken out and 10-20 μL of PCR augmenter was added to the sample in the PCR tube and mixed well by shaking.

TABLE 2
Composition of PCR augmenter (5 ml)

| Chemical | components |
|---|---|
| Triton x100 | 1-2% |
| 2-pyrrolidone | 10 mM |
| Isobutyramide | 2 mM |
| N-methylacetamide | 5 mM |
| Quantum dots | 2 mg/ml |
| Nuclease-free Water | To make the volume to 5 ml |

In an embodiment, after addition of PCR augmenter, 15 μL of PCR reaction medium of table 3 was added to the isolated DNA sample.

TABLE 3
Composition of PCR reaction medium

| Chemical | components |
|---|---|
| DNA | 10-100 ng of genomic DNA |
| Forward Primer | 0.2-1.0 μM |
| Reverse Primer | 0.2-1.0 μM |
| DNA Polymerase | 0.5-2.5 units |
| dNTPs | 200-400 μM each dNTP (dATP, |
| | dCTP, dGTP, dTTP) |
| PCR Buffer | Tris buffer 10 mM pH 7.2 |
| Magnesium Chloride (MgCl2) | 1.5-3.0 mM |
| Nuclease-free Water | To make the volume to 50 μL |

In an embodiment, primers of SEQ ID NO: 1-15 of table 4 are added to the PCR reaction medium.

TABLE 4
sequence of forward primer and reverse primer.

| SEQUENCE | | | Degenerate |
|---|---|---|---|
| ID | Primer Name | Sequence (5′-3′) | Primer |
| SEQ ID NO: 1 | SBFP_1 (Sulphur | CATGGCGCGTTCCGACTTTG | |
| | reducing bacteria) | | |
| SEQ ID NO: 2 | SBRP_1 (Sulphur | CATGCACTTGGAGGGGCACA | |
| | reducing bacteria) | | |
| SEQ ID NO: 3 | SBFP_4 (Sulphur | AGCCGCGGATACTACACGGA | |
| | reducing bacteria) | | |
| SEQ ID NO: 4 | SBRP_4 (Sulphur | TGGTGGTGTCCCCAGCAGTA | |
| | reducing bacteria) | | |
| SEQ ID NO: 5 | AAPB_FP1 (Acid | GCACCGCTTCAYAATCC | Y-C/T |
| | producing | | |
| | microbe) | | |
| SEQ ID NO: 6 | AAPB_RP1 (Acid | GTTTGGTGGAATGCTGTATC | |
| | producing | | |
| | microbe) | | |
| SEQ ID NO: 7 | AAPB_FP2 (Acid | GCACCRCTTCAYAACCC | R-G/A |
| | producing | | |
| | microbe) | | |
| SEQ ID NO: 8 | AAPB_RP2 (Acid | GTTTGATGGAATGCYGTATC | |
| | producing | | |
| | microbe) | | |
| SEQ ID NO: 9 | AAPB_S_F (Acid | CACTGGGTGCGCATAACT | |
| | producing | | |
| | microbe) | | |
| SEQ ID NO: 10 | AAPB_S_R (Acid | ATAACCGAACGGAATCTGGTG | |
| | producing | | |
| | microbe) | | |
| SEQ ID NO: 11 | BAPB_FP1 (Acid | CCATGCATTAAATCAAAAAGC | |
| | producing | | |
| | microbe) | | |
| SEQ ID NO: 12 | BAPB_FP2 | CCATGCGTTAAACCAAAAAGC | |
| SEQ ID NO: 13 | BAPB_RP1 (Acid | AGTACCTCCACCCATGTG | |
| | producing | | |
| | microbe) | | |
| SEQ ID NO: 14 | BAPB_RP2 (Acid | AATACCTCCGCCCATATG | |
| | producing | | |
| | microbe) | | |
| SEQ ID NO: 15 | BAPB_RP3 (Acid | AATACCGCCRCCCATATG | |
| | producing | | |
| | microbe) | | |

20 μl of Positive Control (DNA from known SRB (Sulfur reducing bacteria) and Negative Control (DNA/RNA free water) are added to the PCR tubes using pipette as control reaction condition. The tube was then closed and inserted to portable RT-PCR machine operated under following conditions, as mentioned in Table 5 below:

TABLE 5
RT-PCR conditions for the method of the present disclosure

| Steps | temperature | time | cycles |
|---|---|---|---|
| Initial denaturation | 95° C. | 5 minutes | 1 |
| denaturation | 95° C. | 10 seconds | 40 |
| Annealing/extension& | 52° C. | 20 seconds | 40 |
| Fluorescence | | | |
| acquisition | | | |

*Use SYBR Green channel for Fluorescence acquisition

The microbial content in terms of Colony Forming Units per milliliter (CFU/ml) is determined by using the following formula:

$$Target concentration ( CFU / ml ) = DNA copies corresponding to Sample Ct value \times 800$$

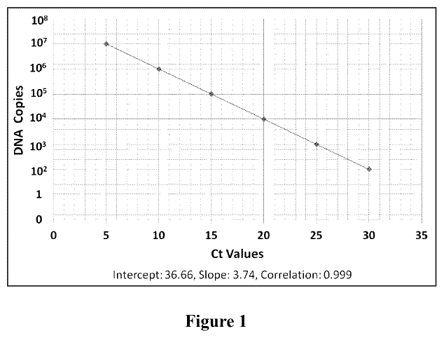

The calculation of DNA copies corresponding to the sample Ct value is performed using the Standard Curve. In an embodiment, the DNA copies corresponding to the Ct value are calculated using the Standard Curve's logarithmic scale graph lines, as provided in FIG. 1 of the present disclosure.

The MIC in various industrial setup is interpreted as follows:
a) SRB=0: No bio-corrosion causing microbes.
b) SRB>10⁵: Minimum to moderate bio-corrosion causing microbes.
c) SRB<10⁵: Severe bio-corrosion causing microbes.

In another aspect of the present disclosure, there is provided a kit for on-site, rapid testing of corrosion-inducing microorganisms. The kit comprises a compact enclosure containing the subsequent constituents:
a) A set of PCR primers of SEQ ID NO: 1-15;
b) A DNA isolation bio-buffer;
c) A PCR augmenter;
d) A PCR Reaction medium;
e) Portable Real-Time Polymerase Chain Reaction (RT-PCR) Apparatus;
f) Pipetting Device;
g) Nano-Scale Sample Analysis System;
h) Mobile Centrifuge Unit;
i) Temperature Regulation Module;
j) Single-use Consumable Items.

Although the subject matter has been described in considerable detail with reference to certain preferred embodiments thereof, other embodiments are possible.

### EXAMPLES

The disclosure will now be illustrated with working examples, which are intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods, the exemplary methods, devices and materials are described herein. It is to be understood that this disclosure is not limited to particular methods, and experimental conditions described, as such methods and conditions may vary.

### Example 1

### Sample Collection:

Samples were collected from the IndianOil Paradip-Haldia-Barauni Oil Pipeline section, India, following the pigging operation.

The collection of sample comprises of following steps:
gathering a sample, preferably a muck sample, from the PIG into a sterile bottle, ensuring no empty space remains; optionally sealing the bottle with Teflon tape if transportation or storage is needed; and labelling the bottles; or
collecting water samples from the drain of the crude storage tank (representative of the bulk phase) directly into sterile bottles, ensuring no empty space remains; optionally sealing the bottle with Teflon tape if transportation or storage is needed; and labelling the bottles; or
for obtaining coupons: position the coupon on aluminum foil and use a scrapper or blade to remove material, placing the scraped material directly into DNA isolation bio-buffer; optionally sealing the bottle with Teflon tape if transportation or storage is needed; and labelling the bottles.

### Example 2: Rapid Identification of MIC Causing Microbes from Crude Transportation Pipeline Using Portable RT-PCR Kit

The muck sample was collected from a pipeline and placed in a closed sterile container. ~0.1 g of muck sample was transferred to 0.5 ml clear PCR tube, followed by brief centrifugation (30 s) such that it settles at the bottom. 100 μL of DNA isolation bio-buffer was added to 0.5 ml clear PCR tube containing muck sample. The composition of DNA isolation bio-buffer is as follows:

TABLE 6
Composition of DNA isolation bio-buffer (5 ml)

| Chemical | components |
|---|---|
| Triton x100 | 1% |
| EDTA | 5-mM |
| TRIS-HCl (pH-8.0) | 10 mM |
| Proteinase K | 20 μg/ml |
| Lysozyme | 0.25 mg/ml |
| CTAB | 2 mg/ml |
| β-Mercaptoethanol | 6 mg/ml |
| Sodium metabisulfite | 3 mg/ml |
| Nuclease-free Water | To make the volume to 5 ml |

After that, 10 μL of PCR augmenter was added to the sample in the PCR tube and mixed well by shaking. The tube was mixed on a dry bath set at 90° C. for 3 minutes to promote sample lysis for release of DNA. The composition PCR augmenter is as follows:

TABLE 7
Composition of PCR augmenter (5 ml)

| Chemical | components |
|---|---|
| Triton x100 | 1% |
| 2-pyrrolidone | 10 mM |
| Isobutyramide | 2 mM |
| N-methylacetamide | 5 mM |
| Carbon quantum dots | 2 mg/ml |
| Nuclease-free Water | To make the volume to 5 ml |

After addition of PCR augmenter, 15 μL of PCR reaction medium was added. The composition of PCR reaction medium is as follows:

TABLE 8
Composition of PCR reaction medium of present method

| Chemical | Components |
|---|---|
| DNA | 90 ng of genomic DNA |
| Forward Primer for SRB | 0.2 μM |
| (CATGGCGCGTTCCGACTTTG) | |
| (SEQ ID NO: 1) | |
| Reverse Primer for SRB | 0.2 μM |
| (CATGCACTTGGAGGGGCACA) | |
| (SEQ ID NO: 2) | |
| DNA Polymerase | 2.5 units |
| dNTPs | 400 μM each dNTP (dATP, |
| | dCTP, dGTP, dTTP) |
| PCR Buffer | Tris buffer 10 mM pH 7.2 |
| Magnesium Chloride (MgCl₂) | 3.0 mM |
| Nuclease-free Water | To make the volume to 50 μL |

20 μL of Positive Control (DNA from known SRB (Sulfur reducing bacteria) and Negative Control (DNA/RNA free water) was introduced to the respective PCR tubes for analysing the control condition vis-à-vis with that of sample. The tube was introduced into the RT-PCR and the steps as shown in below table 9 were followed:

TABLE 9
RT-PCT conditions of the method of the present disclosure

| Steps | temperature | time | cycles |
|---|---|---|---|
| Initial denaturation | 95° C. | 5 minutes | 1 |
| denaturation | 95° C. | 10 seconds | 40 |
| Annealing/extension | 52° C. | 20 seconds | 40 |
| & Fluorescence | | | |
| acquisition | | | |

*Use SYBR Green channel for Fluorescence acquisition

The microbial content in terms of Colony Forming Units per milliliter (CFU/ml) can be determined using the following formula:

$$Target concentration ( CFU / ml ) = DNA copies corresponding to Sample Ct value \times 800.$$

The Ct value was determined to be 10, which corresponds to DNA copies=10⁷.

$$Target concentration ( CFU / ml ) = {10}^{7} * 800 = 8 * {10}^{9}$$

Since SRB<10⁵: it indicates presence of severe bio-corrosion causing microbes.

### Example 3: Quantity of DNA Yield with Time Using DNA Isolation Bio-Buffer Vis-à-Vis Commercial Reagent

The DNA yield was analysed using nanodrop after step-5 of Example 2. The quantity of DNA yield at various time of lysis is given below:

TABLE 10
DNA yields using DNA isolation
bio-buffer vis-à-vis commercial reagent

| | DNA Yield (μg) | |
|---|---|---|
| | DNA isolation bio-buffer | |
| | of composition as the | Commercial reagent |
| Time (Min) | invention | (Composition not reveled) |
| 1 | 0 | 0 |
| 1.5 | 0 | 0 |
| 2 | 0.7 | 0 |
| 2.5 | 3.7 | 0 |
| 3 | 6.3 | 0.7 |
| 3.5 | 6.4 | 2.3 |

### Example 4: Quantification of DNA Stability Before and After Addition of PCR Augmenter

The DNA stability was calculated using the following formula—

DNA stability:(A260−A320)/(A280−A320)

If the ratio is nearer to 2, the stability is higher.

As per example 2 after step-6, the stability was analyzed using a nanodrop as per the above formula and compared with that of commercial samples. The results are shown in Table 11 below:

TABLE 11
Stability of DNA from various samples before and after
addition of PCR augmenter.

| | DNA Stability: (A₂₆₀-A₃₂₀)/(A₂₈₀-A₃₂₀) | |
|---|---|---|
| | Before addition of | After addition of PCR |
| Sample | PCR augmenter | augmenter |
| Muck Sample | 1.5 | 1.9 |
| Pipeline water sample | 1.6 | 1.9 |
| Cooling tower water | 1.2 | 1.9 |
| sample | | |
| Corrosion coupon | 1.7 | 1.9 |

### Example 5: Analysis of Sample from Various Source Using Portable qPCR for MIC Detection Vis-à-Vis Traditional Plating Method

Diverse samples from potential corrosion sites were gathered and exposed to MIC detection using qPCR of the present disclosure. Additionally, the conventional plating technique, as described in existing literature, was also employed. The resulting table 12 below illustrates the concentrations of sulfate-reducing bacteria (SRB) as determined by both approaches.

TABLE 12
Analysis of sample from various source using portable qPCR for
MIC detection vis-à-vis traditional plating method.

| | SRB (CFU) | |
|---|---|---|
| | qPCR based MIC | |
| | detection as per the | |
| Sample | current invention | Plate count method |
| Muck Sample | 10⁹ | 2 ×10⁴ |
| Pipeline water | 10⁷ | 5 × 10³ |
| sample | | |
| Cooling tower water | 10⁴ | 6 × 10² |
| sample | | |
| Corrosion coupon | 10¹⁰ | 4 × 10⁵ |

Hence, the qPCR-based MIC detection, as described in the present disclosure, demonstrates greater precision in forecasting MIC.

### Example 6: Analysis of Sample from Various Source Using Portable qPCR for MIC Detection Vis-à-Vis Traditional Plating Method

Diverse samples from potential corrosion sites were gathered and exposed to MIC detection using qPCR of the present disclosure. Additionally, the conventional plating technique, as described in existing literature, was also employed. The resulting table 13 below illustrates the concentrations of acetic acid producing bacteria (AAPB) and butyric acid producing bacteria (BAPB) as determined by both approaches.

TABLE 13
Analysis of sample from various source using present portable qPCR
for MIC detection vis-à-vis traditional plating method.

| | AABP (Acetic Acid | | BAPB (Butyric Acid | |
|---|---|---|---|---|
| | producing | | Producing | |
| | bacteria) (CFU) | | Bacteria (CFU) | |
| | qPCR based | | qPCR based | |
| | MIC | | MIC | |
| | detection as | | detection as | |
| | per the | | per the | |
| | present | Plate count | present | Plate count |
| Sample | disclosure | method | disclosure | method |
| Muck Sample | 10⁵ | ND | 10³ | ND |
| Pipeline water | 10⁶ | ND | 10³ | ND |
| sample | | | | |
| Cooling tower | 10⁵ | ND | 10³ | ND |
| water sample | | | | |
| Corrosion coupon | 10⁶ | 2.1 × 10² | 10⁴ | ND |

We claim:

1. A set of PCR primers of SEQ ID NO: 1-15 for on-site detection and quantification of corrosion-causing microorganisms.

2. The set of PCR primers as claimed in claim 1, wherein the microorganisms comprise sulfate reducing bacteria (SRB), acetic acid producing bacteria (AAPB) and butyric acid producing bacteria (BAPB).

3. The set of PCR primers as claimed in claim 1, wherein the primers of SEQ ID NO: 1-4 detect and quantify sulfate reducing bacteria (SRB), and wherein the primers of SEQ ID NO: 1 and 3 are forward primers and the primers of SEQ ID NO: 2 and 4 are reverse primers.

4. The set of PCR primers as claimed in claim 1, wherein the primers of SEQ ID NO: 5-10 detect and quantify AAPB, and wherein the primers of SEQ ID NO: 5, 7 and 9 are forward primers and the primers of SEQ ID NO: 6, 8 and 10 are reverse primers.

5. The set of PCR primers as claimed in claim 1, wherein the primers of SEQ ID NO: 11-15 detect and quantify BAPB, and wherein the primers of SEQ ID NO: 11 and 12 are forward primers and the primers of SEQ ID NO: 13, 14 and 15 are reverse primers.

6. An assay based on real-time polymerase chain reaction (RT-PCR) for on-site detection and quantification of corrosion-causing microorganisms from a site affected by corrosion, the assay comprising:
obtaining a sample from the site affected by corrosion;
adding a DNA isolation bio-buffer to isolate DNA from the sample;
stabilizing the isolated DNA with a PCR augmenter;
introducing the stabilized DNA into a PCR reaction medium;
amplifying the stabilized DNA by hybridizing with the primers of SEQ ID NO: 1-15; and
detecting and quantifying the amplified product.

7. The assay as claimed in claim 6, wherein the primers of SEQ ID NO: 1-4 detect and quantify sulfate reducing bacteria (SRB), and wherein the primers of SEQ ID NO: 1 and 3 are forward primers and the primers of SEQ ID NO: 2 and 4 are reverse primers.

8. The assay as claimed in claim 6, wherein the primers of SEQ ID NO: 5-10 detect and quantify AAPB, and wherein the primers of SEQ ID NO: 5, 7 and 9 are forward primers and the primers of SEQ ID NO: 6, 8 and 10 are reverse primers.

9. The assay as claimed in claim 6, wherein the primers of SEQ ID NO: 11-15 detect and quantify BAPB, and wherein the primers of SEQ ID NO: 11 and 12 are forward primers and the primers of SEQ ID NO: 13, 14 and 15 are reverse primers.

10. The assay as claimed in claim 6, wherein the sample is collected from sources comprising muck samples taken during pigging, corrosion coupons, samples from cooling towers, or metallic surfaces found in oil and gas industries, marine industries, infrastructure industries, and water treatment industries.

11. The assay as claimed in claim 6, wherein the microorganisms comprise sulfate reducing bacteria (SRB), acetic acid producing bacteria (AAPB), and butyric acid producing bacteria (BAPB).

12. The assay as claimed in claim 6, wherein the DNA isolation bio-buffer comprises 1-2% TritonX100, 5 mM EDTA, 10 mM Tris HCl, 20 μg/ml Proteinase K, 0.25 mg/ml lysozyme, 2 mg/ml CTAB, 6 mg/ml β-Mercaptoethanol, 3 mg/ml sodium metabisulfite and nuclease free water.

13. The assay as claimed in claim 6, wherein the PCR augmenter comprises 1-2% TritonX100, 10 mM 2-pyrrolidone, 2 mM Isobutyramide, 5 mM N-methylacetamide, 2 mg/ml Quantum dots and nuclease free water.

14. The assay as claimed in claim 6, wherein the PCR reaction medium comprises 90 ng of genomic DNA, 0.2 μM of forward primers, 0.2 μM of reverse primers, 2.5 units of DNA polymerase, 400 μM each dNTP, Tris buffer 10 mM pH 7.2, 3.0 mM of Magnesium Chloride, and nuclease free water to make volume of the sample to 50 μL, wherein each dNTP comprises dATP, dCTP, dGTP, and dTTP, and wherein primers of SEQ ID NO: 1-15 are added to the PCR reaction medium.

15. The assay as claimed in claim 6, further comprising keeping the sample on a dry bath for 7-10 minutes at 90° C. after adding DNA isolation bio-buffer.

16. The assay as claimed in claim 6, wherein the quantification of corrosion-causing microorganisms is performed by a formula
Target concentration(Colony Forming Units per milliliter(CFU/ml))=DNA copies corresponding to cycle threshold value(Ct) of the sample×800,
wherein Ct is the number of PCR cycles required for fluorescence signal to cross a defined threshold, indicating a detectable amount of amplified DNA, wherein 800 is a correlation factor between DNA copies from qPCR) and actual viable microbial count.

17. A kit for on-site detection and quantification of corrosion-causing microorganisms, the kit comprising:
a set of PCR primers comprising of SEQ ID NO: 1-15;
a DNA isolation bio-buffer;
a PCR augmenter;
a PCR reaction medium;
a portable Real-Time Polymerase Chain Reaction (RT-PCR) apparatus;
a nano-scale sample analysis system;
a mobile centrifuge unit; and
a temperature regulation module.

18. The kit as claimed in claim 14, wherein the DNA isolation bio-buffer comprises 1-2% TritonX100, 5 mM EDTA, 10 mM Tris HCl, 20 μg/ml Proteinase K, 0.25 mg/ml lysozyme, 2 mg/ml CTAB, 6 mg/ml β-Mercaptoethanol, 3 mg/ml sodium metabisulfite and nuclease free water.

19. The kit as claimed in claim 14, wherein the PCR augmenter comprises 1-2% TritonX100, 10 mM 2-pyrrolidone, 2 mM Isobutyramide, 5 mM N-methylacetamide, 2 mg/ml Quantum dots and nuclease free water.

20. The kit as claimed in claim 14, wherein the kit is configured for detecting and quantifying corrosion-causing microorganisms, wherein colony forming units per milliliter (CFU/ml) is equal to 0 indicates no corrosion causing microorganisms, CFU/ml>10⁵ indicates presence of minimum to moderate corrosion causing microorganisms, CFU/ml<10⁵ indicates presence of severe corrosion causing microbes.


SEQUENCE LISTING (15 sequences)

SEQ ID NO: 1 - DNA, 20 bp, synthetic construct
  source 1..20: mol_type = other DNA
catggcgcgttccgactttg

SEQ ID NO: 2 - DNA, 20 bp, synthetic construct
  source 1..20: mol_type = other DNA
catgcacttggaggggcaca

SEQ ID NO: 3 - DNA, 20 bp, synthetic construct
  source 1..20: mol_type = other DNA
agccgcggatactacacgga

SEQ ID NO: 4 - DNA, 20 bp, synthetic construct
  source 1..20: mol_type = other DNA
tggtggtgtccccagcagta

SEQ ID NO: 5 - DNA, 17 bp, synthetic construct
  source 1..17: mol_type = other DNA
gcaccgcttcayaatcc

SEQ ID NO: 6 - DNA, 20 bp, synthetic construct
  source 1..20: mol_type = other DNA
gtttggtggaatgctgtatc

SEQ ID NO: 7 - DNA, 17 bp, synthetic construct
  source 1..17: mol_type = other DNA
gcaccrcttcayaaccc

SEQ ID NO: 8 - DNA, 20 bp, synthetic construct
  source 1..20: mol_type = other DNA
gtttgatggaatgcygtatc

SEQ ID NO: 9 - DNA, 18 bp, synthetic construct
  source 1..18: mol_type = other DNA
cactgggtgcgcataact

SEQ ID NO: 10 - DNA, 21 bp, synthetic construct
  source 1..21: mol_type = other DNA
ataaccgaacggaatctggtg

SEQ ID NO: 11 - DNA, 21 bp, synthetic construct
  source 1..21: mol_type = other DNA
ccatgcattaaatcaaaaagc

SEQ ID NO: 12 - DNA, 21 bp, synthetic construct
  source 1..21: mol_type = other DNA
ccatgcgttaaaccaaaaagc

SEQ ID NO: 13 - DNA, 18 bp, synthetic construct
  source 1..18: mol_type = other DNA
agtacctccacccatgtg

SEQ ID NO: 14 - DNA, 18 bp, synthetic construct
  source 1..18: mol_type = other DNA
aatacctccgcccatatg

SEQ ID NO: 15 - DNA, 18 bp, synthetic construct
  source 1..18: mol_type = other DNA
aataccgccrcccatatg